SEQUENCE LISTING (28 sequences)

SEQ ID NO: 1 - DNA, 70 bp, Artificial Sequence
  Description of Artificial Sequence Synthetic oligonucleotide
ctctctcatcagcgaaccacaactcaagacctcgttaagggagcggagcggtaatgctagttattgtcca

SEQ ID NO: 2 - DNA, 50 bp, Artificial Sequence
  Description of Artificial Sequence Synthetic oligonucleotide
ctctctcatcagcgaaccacaactcaagacctcgttaagggagcggagcg

SEQ ID NO: 3 - DNA, 20 bp, Artificial Sequence
  Description of Artificial Sequence Synthetic probe
cgctccgctcccttaacgag

SEQ ID NO: 4 - DNA, 20 bp, Artificial Sequence
  Description of Artificial Sequence Synthetic probe
tggacaataactagcattac

SEQ ID NO: 5 - DNA, 25 bp, Artificial Sequence
  Description of Artificial Sequence Synthetic primer
atccggatatagttcctcctttcag

SEQ ID NO: 6 - DNA, 21 bp, Artificial Sequence
  Description of Artificial Sequence Synthetic primer
ccatcgccgcttccacttttt

SEQ ID NO: 7 - DNA, 22 bp, Artificial Sequence
  Description of Artificial Sequence Synthetic probe
ccagtagtaggttgaggccgtt

SEQ ID NO: 8 - DNA, 22 bp, Artificial Sequence
  Description of Artificial Sequence Synthetic probe
gactcctgcattaggaagcagc

SEQ ID NO: 9 - DNA, 840 bp, Artificial Sequence
  Description of Artificial Sequence Synthetic polynucleotide
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgcgacccatttgctgtccaccagtcatgctagccatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatgg

SEQ ID NO: 10 - DNA, 60 bp, Artificial Sequence
  Description of Artificial Sequence Synthetic oligonucleotide
aaaaaaaaaaaaaaaaaaaaaaaaaaaaaaaaaaaaaaaaaaaaaaaaaaaaaaaaaaaa

SEQ ID NO: 11 - DNA, 20 bp, Artificial Sequence
  Description of Artificial Sequence Synthetic oligonucleotide
tttttttttttttttttttt

SEQ ID NO: 12 - DNA, 24 bp, Artificial Sequence
  Description of Artificial Sequence Synthetic primer
gaccagtaagttcagagatgcaga

SEQ ID NO: 13 - DNA, 22 bp, Artificial Sequence
  Description of Artificial Sequence Synthetic primer
caaccagaccaggtagacagag

SEQ ID NO: 14 - DNA, 20 bp, Artificial Sequence
  Description of Artificial Sequence Synthetic probe
tgttctcaccgatacacttc

SEQ ID NO: 15 - DNA, 20 bp, Artificial Sequence
  Description of Artificial Sequence Synthetic probe
aaagacctcccagcggccaa

SEQ ID NO: 16 - DNA, 530 bp, Artificial Sequence
  Description of Artificial Sequence Synthetic polynucleotide
gaccagtaagttcagagatgcagaggaaaggctgggtccaccctcttaagctcttatatatgattaatacaatcattgcattgatcctcctgtccatgggctgcttgcctgtcctctatcctttggggctggagctccactcacttgacacttctgagccctgaactgccacttcagctgtctccctctggttacaggaagctatgggtcaacccatctgagttcctacctgaacggtttctcacccctgatggtgctatcgacaaggtgttaagtgagaaggtgattatctttggcatgggcaagcggaagtgtatcggtgagaacrttggccgctgggaggtctttctcttcctggctatcctgctgcaacgggtggaattcagcgtgccactgggcgtgaaggtggacatgacccccatctatgggctaaccatgaagcatgcctgctgtgagcacttccaaatgcagctgcgctcttaggtgcttgagagccctgaggcctagactctgtctacctggtctggttg

SEQ ID NO: 17 - DNA, 20 bp, Artificial Sequence
  Description of Artificial Sequence Synthetic primer
tgtgtgacctaactgtgtaa

SEQ ID NO: 18 - DNA, 20 bp, Artificial Sequence
  Description of Artificial Sequence Synthetic primer
accttcccactagagcttgg

SEQ ID NO: 19 - DNA, 30 bp, Artificial Sequence
  Description of Artificial Sequence Synthetic probe
taatctattacactttatattacccattat

SEQ ID NO: 20 - DNA, 30 bp, Artificial Sequence
  Description of Artificial Sequence Synthetic probe
ggtttcttttctctctcccacccacaacta

SEQ ID NO: 21 - DNA, 259 bp, Artificial Sequence
  Description of Artificial Sequence Synthetic polynucleotide
tgtgtgacctaactgtgtaatttcactgaagaaaccttcagatttgtgttataatgggtaatataaagtgtaatagattatagttgtgggtgggagagagaaaagaaaccagagtccaaatttgaaaataattgttccaaatgaatgttgtctgtttgttctctcttagtaacatagacaaaaaatttgagaaagccttcataagcctaccaatgtagacacgctcttctattttattcccaagctctagtgggaaggt

SEQ ID NO: 22 - DNA, 24 bp, Artificial Sequence
  Description of Artificial Sequence Synthetic primer
aagcgcacgctgcggaggctgctg

SEQ ID NO: 23 - DNA, 20 bp, Artificial Sequence
  Description of Artificial Sequence Synthetic primer
ggctgccaggtcgcggtgca

SEQ ID NO: 24 - DNA, 30 bp, Artificial Sequence
  Description of Artificial Sequence Synthetic probe
gcttctcttaattccttgatagcgacggga

SEQ ID NO: 25 - DNA, 31 bp, Artificial Sequence
  Description of Artificial Sequence Synthetic probe
gaggatttccttgttggctttcggagatgtt

SEQ ID NO: 26 - DNA, 495 bp, Artificial Sequence
  Description of Artificial Sequence Synthetic polynucleotide
aagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagcc

SEQ ID NO: 27 - DNA, 40 bp, Artificial Sequence
  Description of Artificial Sequence Synthetic oligonucleotide
aaaaaaaaaaaaaaaaaaaaaaaaaaaaaaaaaaaaaaaa

SEQ ID NO: 28 - DNA, 80 bp, Artificial Sequence
  Description of Artificial Sequence Synthetic oligonucleotide
aaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2010/0167281 A1
(43) Pub. Date: Jul. 1, 2010

(54) SEQUENCE ANALYSIS METHOD

(76) Inventors: Maiko Tanabe, Tokyo (JP); Chihiro Uematsu, Kawasaki (JP); Hirokazu Nishida, Kokubunji (JP); Kenko Uchida, Tokyo (JP)

Correspondence Address: MATTINGLY & MALUR, P.C., 1800 DIAGONAL ROAD, SUITE 370, ALEXANDRIA, VA, 22314, US

(21) Appl. No.: 12/389,151

(22) Filed: Feb. 19, 2009

(30) Foreign Application Priority Data
Jul. 30, 2008 (JP) 2008-196782

(51) Int. Cl. C12Q 1/68

(52) U.S. Cl. 435/6

(57) ABSTRACT

It is intended to provide an assay for the presence, absence or amount of a nucleic-acid fragment having a certain nucleotide sequence, for example, a polyA length, a difference in the number of repetition of a direct repeat sequence (e.g., microsatellite), single nucleotide substitution (or single nucleotide polymorphism), and nucleotide sequence insertion or deletion, and to provide a genetic testing using the same. The present invention relates to a nucleotide analysis method, comprising: hybridizing at least two probes to a nucleic-acid fragment; ligating the at least two probes using ligase; exchanging, to ATP, pyrophosphoric acid produced through the ligation reaction; and detecting chemiluminescence reaction dependent on the ATP.

### CLAIM OF PRIORITY

The present application claims priority from Japanese Patent application JP 2008-196782 filed on Jul. 30, 2008, the content of which is hereby incorporated by reference into this application.

### BACKGROUND OF THE INVENTION

1. Field of the Invention

The present invention relates to a sequence analysis method for a nucleic acid sample, which is useful in genome analysis. This method comprises analyzing a nucleic acid sequence by qualitatively and quantitatively detecting pyrophosphoric acid produced in response to ligation reaction of nucleic acids. More specifically, the present invention relates to an assay for determining the presence or absence or amount of a nucleic-acid fragment having a certain nucleotide sequence, for example, a polyA length, a difference in the number of repetition of a direct repeat sequence (e.g., microsatellite), single nucleotide substitution (or single nucleotide polymorphism), and nucleotide sequence insertion or deletion, and to a genetic testing using the same.

2. Background Art

With the completion of the human genome sequencing, many characteristic nucleotide sequences have been reported, which serve as markers for disease, drug responsiveness, or acquiring disease. Such markers are mainly a polyA length, a difference in the number of repetition of a direct repeat sequence (e.g., microsatellite), single nucleotide substitution (or single nucleotide polymorphism; hereinafter, SNP), and nucleotide sequence insertion/deletion. Some characteristic nucleotide sequences have already been approved as genetic markers for diagnostic use by FDA (Food and Drug Administration). Such nucleotide sequences are detected mainly according to methods including: the dideoxy method (Sanger method) which involves elongating a sequence of interest through elongation reaction catalyzed by DNA polymerase and analyzing the nucleotide sequence (F. Sanger et al., Journal of Molecular Biology, 94, 411-448 (1975)); the DNA microarray method which involves detecting a mutation on the DNA chip by hybridizing with target sequence (J. G. Hacia et al., Nat Genet, 22, 164-167 (1999)); and the Invader assay which involves detecting nucleotide substitution using an enzyme that recognizes a single nucleotide difference (M. Arruda et al., Expert Review of Molecular Diagnostics, 2, 487-496 (2002)).

All of these analysis methods are convenient and have been verified as promising approaches. The dideoxy method can analyze characteristic nucleotide sequences other than polyA and has, however, limitation in a base length that can be analyzed at a time. The microarray method or the Invader assay can analyze a genome size and is, however, incapable of analyzing characteristic nucleotide sequences other than SNP. Thus, disadvantageously, none of these methods can analyze a polyA length, a difference in the number of repetition of a direct repeat sequence, or nucleotide sequence insertion/deletion without limitation in a base length to be analyzed. Other analysis methods used include: PCR (polymerase chain reaction) (R. K. Saiki, et al., Science, 239, 487-491 (1988)) used as general nucleic acid amplification; its applications PCR-SSCP (single-strand conformation polymorphism) (K. Hayashi et al., PCR Methods Appl, 1, 34-38 (1991)) and STR-PCR (C. P. Kimpton et al., PCR Methods Appl, 3, 13-22 (1993)); and the poly(A) test which involves DNA joining reaction (F. J. Salles et al., Genome Res, 4, 317-321 (1995)). All of these methods require the procedure of separating a sample for detection by electrophoresis after reaction. Therefore, their complicated detection procedures are disadvantageous.

### SUMMARY OF THE INVENTION

An object of the present invention is to solve the problems of conventional sequence analysis methods and to provide a method for conveniently conducting the qualitative judgment and quantitative detection of a sequence of interest in a nucleic acid sample, more specifically, the detection of a polyA length, the number of repetition of a direct repeat sequence (e.g., microsatellite), SNP, and nucleotide sequence insertion/deletion, without limitation in a base length to be analyzed.

The present inventors have completed the present invention by finding that a polyA length, a difference in the number of repetition of a direct repeat sequence, and nucleotide sequence insertion/deletion can be detected conveniently without limitation in a base length to be analyzed, by: hybridizing at least two probes to a nucleic-acid fragment; ligating the at least two probes using ligase; exchanging, to ATP, pyrophosphoric acid produced through the ligation reaction; and detecting chemiluminescence reaction dependent on the ATP.

Specifically, the present invention encompasses the followings:

(1) A nucleotide analysis method, comprising: hybridizing at least two probes to a nucleic-acid fragment; ligating the at least two probes using ligase; exchanging, to ATP, pyrophosphoric acid produced through the ligation reaction; and detecting chemiluminescence reaction dependent on the ATP.
(2) The nucleotide analysis method according to (1), wherein the at least two probes are hybridized to adjacent regions, respectively, in the nucleic-acid fragment.
(3) The nucleotide analysis method according to (1), wherein at least one probe of the at least two probes has a 5′-end labeled with a phosphate group.
(4) The nucleotide analysis method according to (1), wherein the ligase catalyzes the ligation reaction using a substrate, and the chemiluminescence reaction is catalyzed by luciferase, wherein the substrate is substantially unreactive with the luciferase.
(5) The nucleotide analysis method according to (1), wherein the ligase is capable of catalyzing the ligation reaction using the substrate which is substantially unreactive with the luciferase.
(6) The nucleotide analysis method according to (1), wherein the chemiluminescence reaction is detected to thereby detect the presence, absence and/or amount of the sequence of interest in the nucleic-acid fragment.
(7) The nucleotide analysis method according to (1), wherein the at least two probes are hybridized to RNA or DNA sequence regions, respectively, in the nucleic-acid fragment.
(8) The nucleotide analysis method according to (1), wherein the at least two probes are hybridized to an amplified nucleic-acid fragment as the nucleic-acid fragment.
(9) The nucleotide analysis method according to (1), wherein the at least two probes each comprise an oligo dT nucleotide.
(10) The nucleotide analysis method according to (9), wherein the chemiluminescence reaction is detected to thereby measure the length of the nucleic-acid fragment.
(11) The nucleotide analysis method according to (1), wherein the at least two probes are hybridized to direct repeat sequence regions, respectively, in the nucleic-acid fragment.
(12) The nucleotide analysis method according to (11), wherein the direct repeat sequence in the nucleic-acid fragment is a particular nucleotide sequence occurring repetitively.
(13) The nucleotide analysis method according to (11), wherein the at least two probes each comprise a complementary sequence to the direct repeat sequence.
(14) The nucleotide analysis method according to (11), wherein the chemiluminescence reaction is detected to thereby measure the number of repetition of the direct repeat sequence.
(15) The nucleotide analysis method according to (1), wherein at least one probe of the at least two probes has an end corresponding to an SNP site in the nucleic-acid fragment.
(16) The nucleotide analysis method according to (15), wherein the chemiluminescence reaction is detected to thereby determine the presence or absence of the ligation reaction, based on which the presence or absence of a mutation in the SNP site is determined.
(17) The nucleotide analysis method according to (1), wherein the at least two probes are hybridized to regions flanking upstream and downstream of a nucleotide sequence insertion site, respectively, in the nucleic-acid fragment.
(18) The nucleotide analysis method according to (17), wherein the chemiluminescence reaction is detected to thereby determine the presence or absence of the ligation reaction, based on which the presence or absence of a mutation in the nucleotide sequence insertion site is determined.
(19) The nucleotide analysis method according to (1), wherein at least one probe of the at least two probes has an end corresponding to a nucleotide sequence deletion site in the nucleic-acid fragment.
(20) The nucleotide analysis method according to (19), wherein the chemiluminescence reaction is detected to thereby determine the presence or absence of the ligation reaction, based on which the presence or absence of a mutation in the nucleotide sequence deletion site is determined.

The present invention achieves the convenient detection of the presence/absence or amount of a sequence of interest in a nucleic acid sample without limitation in a base length to be analyzed. The method of the present invention can also conveniently detect a polyA length, the number of repetition of a direct repeat sequence, and mutations such as SNP or nucleotide sequence insertion/deletion.

### BRIEF DESCRIPTION OF THE DRAWINGS

FIG. 1 is a diagram showing a first embodiment of the present invention.

FIG. 2 is a diagram showing a second embodiment of the present invention.

FIG. 3 is a diagram showing the details of the second embodiment of the present invention.

FIG. 4 is a diagram showing a third embodiment of the present invention.

FIG. 5 is a diagram showing a fourth embodiment of the present invention.

FIG. 6 is a diagram showing a fifth embodiment of the present invention.

FIG. 7 is a diagram showing a sixth embodiment of the present invention.

FIG. 8 is a diagram showing an embodiment of a reaction flow according to the present invention.

FIG. 9 is a diagram showing electrophoresis analysis results of a reaction product according to the first embodiment of the present invention.

FIG. 10 is a diagram showing an embodiment of a reaction flow according to the present invention.

FIG. 11 is a diagram showing chemiluminescence detection results of a reaction product according to the first embodiment of the present invention.

FIG. 12 shows the nucleotide sequence of a nucleic acid sample used in Example 3.

FIG. 13 is a diagram showing an embodiment of a reaction flow according to the present invention.

FIG. 14 is a diagram showing chemiluminescence detection results of a reaction product according to the second embodiment of the present invention.

FIG. 15 is a diagram showing an embodiment of a reaction flow according to the present invention.

FIG. 16 is a diagram showing chemiluminescence detection results of a reaction product according to the third embodiment of the present invention.

FIG. 17 shows the nucleotide sequence of a nucleic acid sample used in Example 5.

FIG. 18 is a diagram showing chemiluminescence detection results of a reaction product according to the fourth embodiment of the present invention.

FIG. 19 shows the nucleotide sequence of a nucleic acid sample used in Example 6.

FIG. 20 is a diagram showing chemiluminescence detection results of a reaction product according to the fifth embodiment of the present invention.

FIG. 21 shows the nucleotide sequence of a nucleic acid sample used in Example 7.

FIG. 22 is a diagram showing chemiluminescence detection results of a reaction product according to the sixth embodiment of the present invention.

### DESCRIPTION OF SYMBOLS

1, 10, 30, 31, 40, 41, 50, 51, 60, and 61 . . . nucleic acid sample
2, 3, 11, 12, 13, 14, 32, 42, 43, 53, 54, 55, 62, 63, and 64 . . . nucleotide sequence in nucleic acid sample
4, 5, 19, 20, 33, 44, 45, 56, 57, 67, 68, and 127 . . . probe
6, 21, 34, 35, 46, 58, and 69 . . . ligation product
15 and 16 . . . primer
17 and 18 . . . elongation product
65 and 66 . . . probe nucleotide sequence
80, 95, and 125 . . . reaction solution containing nucleic acid sample, ligase, luminescent reagent, ligase substrate, and reaction reagent
81, 97, and 115 . . . probe mixture solution
82 . . . reaction device
83 . . . detection device
90 . . . electrophoresis image
91, 92, and 93 . . . band
96, 114, and 126 . . . reaction/detection device
100, 120, 130, 140, 150, and 160 . . . luminescence spectrogram
101, 121, 122, 131, 132, 133, 141, 142, 151, 152, 161, and 162 . . . luminescence spectrum
105, 135, 145, and 155 . . . nucleotide sequence of nucleic acid sample
110 . . . reaction solution containing nucleic acid sample, polymerase, and PCR reaction reagent
111 . . . amplification reaction device
112 . . . elongation product
113 . . . reaction solution containing ligase, luminescent reagent, ligase substrate, and reaction reagent

### DETAILED DESCRIPTION OF THE PREFERRED EMBODIMENTS

The present inventors have developed an analysis method that can conveniently detect the presence/absence and amount of a sequence of interest contained in a nucleic acid sample. In the present invention, at least two probes each having a complementary sequence to a nucleic acid sequence of interest are hybridized to a nucleic acid sample (nucleic-acid fragment); the at least two probes are ligated using ligase; pyrophosphoric acid produced as a result of the ligation is exchanged to ATP; and the amount of chemiluminescence generated by luciferase can be detected to thereby analyze the presence/absence and amount of the nucleic acid sequence in the nucleic acid sample. Alternatively, for the detection of a polyA length, the number of repetition of a direct repeat sequence, SNP, or nucleotide sequence insertion/deletion, poly dT oligonucleotide probes or probes complementary to the direct repeat sequence or to a sequence containing the mutation site and regions adjacent thereto are utilized. Chemiluminescence reaction can be detected in the same way as above to thereby analyze the sequence length, the number of repetition, or the presence or absence of the mutation.

In the present invention, at least two probes, i.e., plural probes are used. The sequences of the at least two probes are usually designed such that they are hybridized to adjacent regions, respectively, in the nucleic-acid fragment. At least one of the two probes which are hybridized to adjacent regions, respectively, in the nucleic-acid fragment usually has a 5′-end labeled with a phosphate group. This 5′-end labeled with a phosphate group is ligated with the 3′-end of the other probe using ligase.

The ligase, preferably, DNA ligase, catalyzes the ligation reaction using a substrate. Specifically, the ligase is capable of incorporating the substrate (ligase substrate) and catalyzing the ligation reaction in this state. Preferable examples of the ligase include ATP-dependent DNA ligase, e.g., archaeal DNA ligase (Pfu DNA ligase, KOD DNA ligase, etc).

The ligase substrate is, preferably, substantially unreactive with the luciferase. Specifically, the ligase substrate is, preferably, substantially unreactive with the luciferase, while being capable of serving as a substrate for the ligation reaction catalyzed by the ligase. Preferable examples of the ligase substrate include ATP analogues, e.g., dATP and labeled-a-phosphate-containing ATP analogues (ATPαS and dATPαS). In this context, the term “substantially” means that the reactivity stays at 0.25 or lower, more preferably 1.0×10⁻⁴ or lower (corresponding to that of dATPαS), when the amount of luminescence generated by the luciferase in the presence of ATP is defined as 1. Thus, the ligase is preferably ligase such as Pfu DNA ligase, whose substrate can be a substrate which is substantially unreactive with the luciferase used in the chemiluminescence reaction, for example, ATP analogues (e.g., dATPαS).

The chemiluminescence reaction is usually catalyzed by luciferase. This chemiluminescence reaction which is catalyzed by luciferase is known as a method for rapid and highly sensitive ATP measurement and also called luciferin/luciferase reaction. This reaction is dependent on ATP. Luciferin reacts with ATP to form luciferyl adenylate. This luciferyl adenylate undergoes degradation through oxidative decarboxylation with oxygen in the presence of luciferase. A portion of energy obtained during this reaction appears as luminescence reaction. This luminescence can be quantified to thereby quantify the ATP.

Pyrophosphoric acid (PPi) produced as a result of the probe ligation catalyzed by the ligase is exchanged to ATP by ATP synthase. Chemiluminescence dependent on the produced ATP is detected in the presence of luciferase that catalyzes the chemiluminescence reaction with the ATP as a substrate.

ATP sulfurylase, pyruvate phosphate dikinase (hereinafter, PPDK), or phenylalanine racemase can be used as the ATP synthase that catalyzes the ATP production from the pyrophosphoric acid. Moreover, the sequence of the nucleic-acid fragment (i.e., nucleic acid sample) may be any of DNA and RNA sequences. Both single strand and double strand DNAs can be analyzed. The double strand DNA, when used as a template, may be denatured into single strands in a pretreatment step and then subjected to the method of the present invention. Alternatively, RNA produced by a reverse transcription reaction can also be analyzed by the method of the present invention. A trace amount of DNA can be used in the form of an elongation product amplified through PCR reaction (amplified nucleic-acid fragment). A trace amount of mRNA can be used in the form of a reaction product according to the PCR-based oligo(G)-tailing method (Y. Y. Kusov et al., Nucleic Acids Res, 29, e57 (2001)).

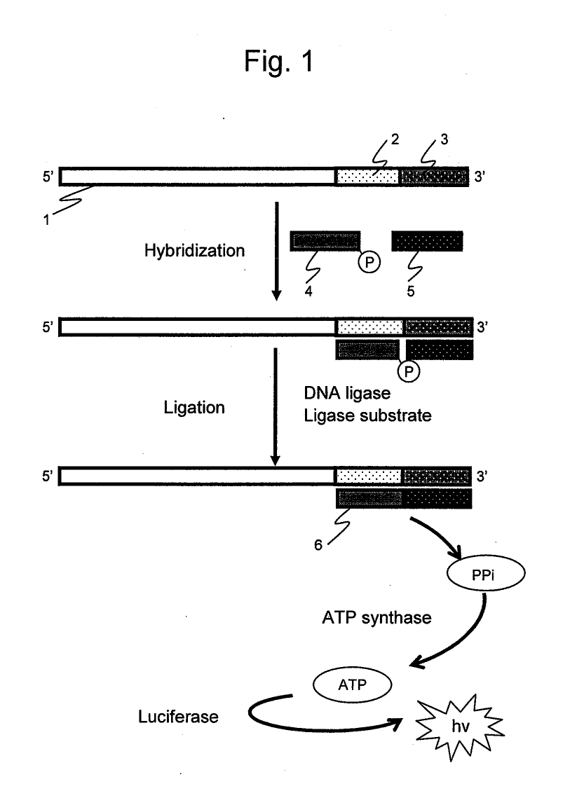

A first embodiment of the present invention is shown in FIG. 1. The present embodiment relates to a nucleotide analysis method comprising: a first step of hybridizing, to a single strand nucleic acid sample 1, a probe 4 which has a complementary sequence to a nucleic acid sequence 2 present in the single strand nucleic acid sample 1 and has a 5′-end labeled with a phosphate group and a probe 5 which has a complementary sequence to a nucleic acid sequence 3 present in the single strand nucleic acid sample 1; a second step of ligating the probes hybridized in the first step using ligase incorporating a ligase substrate to obtain a ligation product 6; a third step of exchanging pyrophosphoric acid produced as a result of the second step to ATP using ATP synthase; and a step of detecting chemiluminescence dependent on the obtained ATP (preferably, luminescence generated through chemiluminescence reaction catalyzed by luciferase with the ATP as a substrate). More specifically, the present embodiment relates to a method for detecting the presence or absence of the sequence of interest in the nucleic acid sample.

FIG. 1 shows an embodiment wherein the nucleic acid sequences 2 and 3 are present in the nucleic acid sample. In this case, in the first step, both the probes 4 and 5 are hybridized adjacently to each other to the nucleic acid sample. Therefore, in the second step, ligation takes place between these probes using ligase to obtain the ligation product 6. Accordingly, pyrophosphoric acid is produced, and chemiluminescence is detected. However, the nucleic acid sequence 2 or 3 may be absent in the nucleic acid sample. In this case, in the first step, the probe 4 or 5 fails to be hybridized thereto. Therefore, ligation by ligase does not take place. Accordingly, no pyrophosphoric acid is produced, and chemiluminescence is not detected. Based on the presence or absence of this detectable chemiluminescence, the presence or absence of the nucleic acid sequence in the nucleic acid sample can be detected. The probes 4 and 5 are designed such that they can be hybridized to the nucleic acid sequences 2 and 3, respectively, present in the nucleic acid sample and ligated in this hybridized state by the ligase. Specifically, the nucleic acid sequences 2 and 3 are adjacent to each other, and the probes 4 and 5, when hybridized thereto, become adjacent to each other.

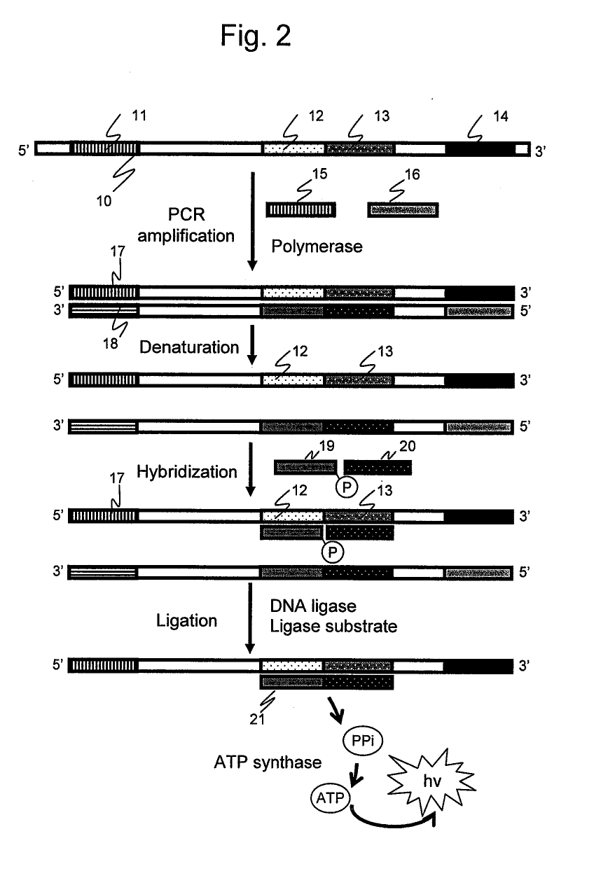

A second embodiment of the present invention is shown in FIG. 2. The present embodiment relates to a nucleotide analysis method comprising: a first step of performing PCR reaction catalyzed by polymerase using a primer 15 which has the same sequence as a nucleic acid sequence 11 present in a single strand nucleic acid sample 10 and a primer 16 which has a complementary sequence to a nucleic acid sequence 14 therein; a second step of denaturing, into single strands, a primer 15-derived elongation product 17 and a primer 16-derived elongation product 18 obtained in the first step; a third step of hybridizing, to the elongation product 17, a probe 19 which has a complementary sequence to a nucleic acid sequence 12 present in the nucleic acid sample 10 and the elongation product 17 and has a 5′-end labeled with a phosphate group and a probe 20 which has a complementary sequence to a nucleic acid sequence 13 therein; a fourth step of ligating the hybridized probes using ligase incorporating a ligase substrate to obtain a ligation product 21; a fifth step of exchanging pyrophosphoric acid produced as a result of the fourth step to ATP using ATP synthase; and a step of qualitatively or quantitatively detecting chemiluminescence dependent on the obtained ATP. More specifically, the present embodiment relates to a method for detecting the sequence of interest in the nucleic acid sample and quantifying the amount thereof. In the second embodiment of the present invention, the probes are hybridized to an elongation product obtained through amplification reaction (amplified nucleic-acid fragment) to cause ligation reaction.

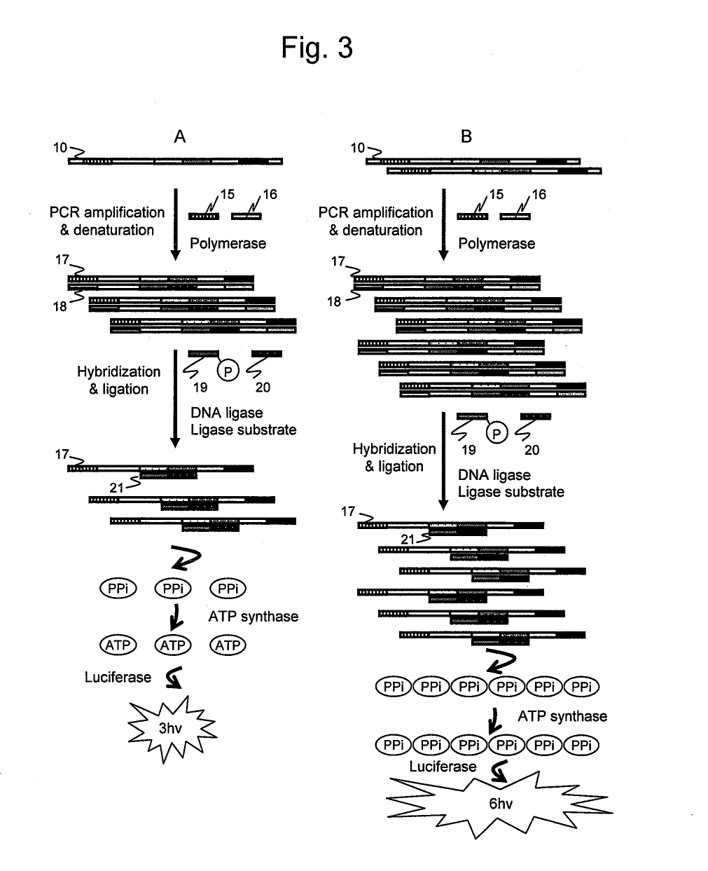

FIG. 3A shows the analysis of one copy of the single strand nucleic acid sample 10. FIG. 3B shows the analysis of two copies of the single strand nucleic acid sample 10. Under the same reaction conditions, the copy number of the elongation product (amplified nucleic-acid fragment) obtained in the first step depends on the copy number of the nucleic acid sample before amplification. The elongation product 17 is obtained in 3 copies in FIG. 3A, while the elongation product 17 is obtained in 6 copies in FIG. 3B. In the second embodiment of the present invention, luminescence intensity detected is proportional to the number of ligated sites between the probes, i.e., the number of the ligation product 21 obtained in the fourth step. The number of the ligation product 21 is proportional to the copy number of the elongation product 17. For easy understanding, luminescence intensity obtained for one ligation reaction is defined as 1 hv. FIG. 3A shows 3 copies of the elongation product 17, from which three ligation products 21 are in turn obtained, resulting in the detected luminescence intensity of 3 hv. On the other hand, FIG. 3B shows 6 copies of the elongation product 17, from which six ligation products 21 are in turn obtained, resulting in the detected luminescence intensity of 6 hv. Thus, the amount of chemiluminescence derived from the ligation product obtained depending on the amount of the elongation product can be detected to thereby conveniently quantify the nucleic acid sample.

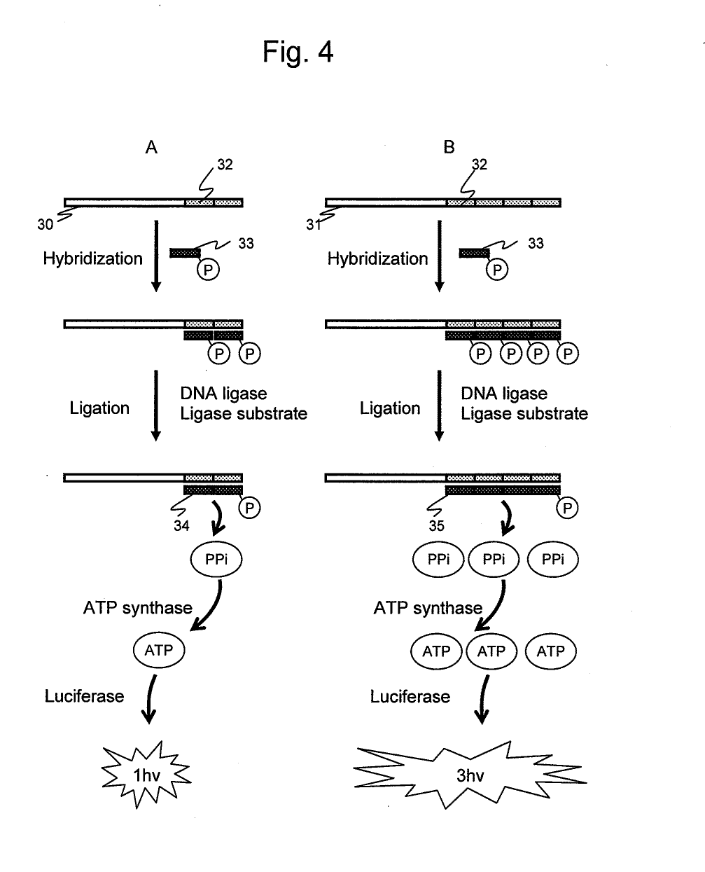

A third embodiment of the present invention is shown in FIG. 4. The present embodiment shows that probes are hybridized to direct repeat sequence (particular nucleotide sequence occurring repetitively) regions, respectively, in a nucleic-acid fragment. In this case, the probes usually respectively comprise a complementary sequence to the direct repeat sequence. The present embodiment relates to a nucleotide analysis method comprising: a first step of hybridizing, to a single strand nucleic acid sample (30 or 31), each probe 33 which has a complementary sequence to a direct repeat sequence 32 present in the single strand nucleic acid sample 30 or 31 and has a 5′-end labeled with a phosphate group; a second step of ligating the probes 33 hybridized in the first step using ligase incorporating a ligase substrate; a third step of exchanging pyrophosphoric acid produced as a result of the second step to ATP using ATP synthase; and a step of quantitatively detecting chemiluminescence dependent on the obtained ATP. More specifically, the present embodiment relates to a method for measuring the number of repetition of the direct repeat sequence in the nucleic acid sample. The single strand nucleic acid samples 30 and 31 used in this analysis must indispensably have equal molar concentrations. The number of repetition can be measured to thereby measure even the length of the nucleic acid sample.

FIG. 4A shows the analysis of the single strand nucleic acid sample 30 having the direct repeat sequence 32 repeated twice. FIG. 4B shows the analysis of the single strand nucleic acid sample 31 having the direct repeat sequence 32 repeated four times. Luminescence intensity detected is proportional to the number of ligated sites between the probes, i.e., the number of the pyrophosphoric acid obtained by the ligation. FIG. 4A shows one ligated site in the ligation product 34 obtained in the second step, from which one pyrophosphoric acid is in turn obtained, resulting in the detected luminescence intensity of 1 hv. On the other hand, FIG. 4B shows three ligated sites in the ligation product 35 obtained in the second step, from which pyrophosphoric acid three times that obtained from one ligated site is in turn obtained, resulting in the detected luminescence intensity of 3 hv. Thus, the amount of chemiluminescence obtained depending on the amount of pyrophosphoric acid produced as a result of the probe ligation can be detected to thereby conveniently detect the number of repetition of the direct repeat sequence in the nucleic acid sample. The probe 33 used in the present embodiment may comprise plural repetitive sequences. Moreover, a poly dT oligonucleotide sequence can be used as the probe 33 to thereby analyze a polyA length.

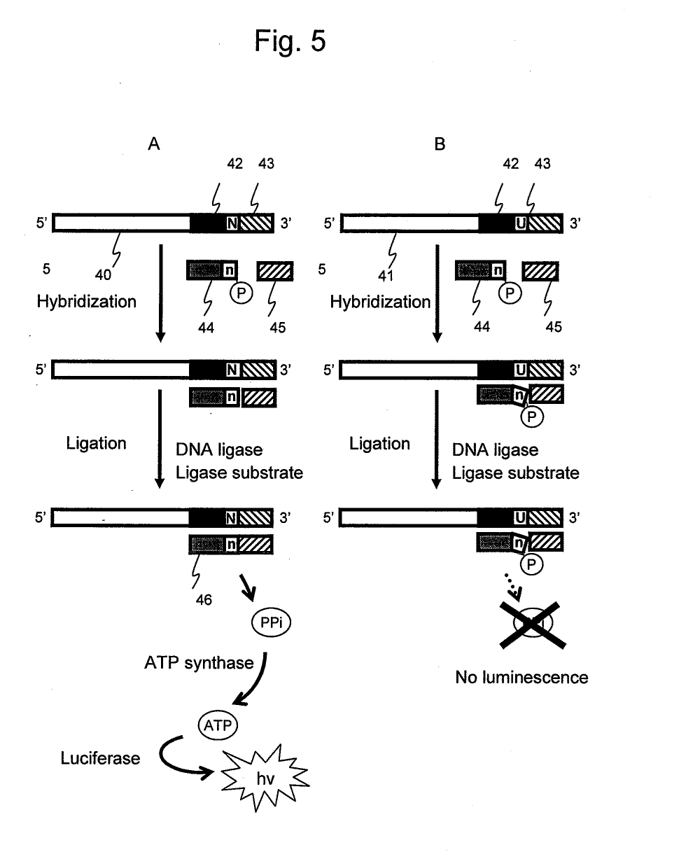

A fourth embodiment of the present invention is shown in FIG. 5. The present embodiment shows that at least one probe of at least two probes has an end (5′- or 3′-end) corresponding to (complementary to) an SNP site in a nucleic-acid fragment (nucleic acid sample). The present embodiment relates to a nucleotide analysis method comprising: a first step of hybridizing, to a single strand nucleic acid sample 40 containing one SNP nucleotide N between nucleic acid sequences 42 and 43 or a single strand nucleic acid sample 41 containing one SNP nucleotide U therebetween, a probe 44 which contains nucleotide n complementary to the one SNP nucleotide N at its 5′-end labeled with a phosphate group and has a complementary sequence to the nucleic acid sequence 42 and a probe 45 which has a complementary sequence to the nucleic acid sequence 43; a second step of performing ligation reaction of the probes hybridized in the first step using ligase incorporating a ligase substrate; a third step of exchanging pyrophosphoric acid produced through the successful ligation reaction to ATP using ATP synthase; and a step of detecting chemiluminescence dependent on the obtained ATP. More specifically, the present embodiment relates to a method for detecting SNP (the presence or absence of a mutation in the SNP site) in the nucleic acid sample.

FIG. 5A shows the analysis of the single strand nucleic acid sample 40 containing the SNP site N free from a mutation. FIG. 5B shows the analysis of the single strand nucleic acid sample 41 containing the mutated SNP site U. In FIG. 5A that shows the mutation-free SNP site, a ligation product 46 is obtained in the second step. Accordingly, pyrophosphoric acid is produced, and chemiluminescence is detected. On the other hand, in FIG. 5B that shows the mutated SNP site, this SNP site takes a single strand form due to a mismatch between the site and the 5′-end n of the probe hybridized in the first step. Therefore, ligation does not take place using ligase in the second step. Accordingly, no pyrophosphoric acid is produced, and chemiluminescence is not detected. Based on the presence or absence of this detectable chemiluminescence, the presence or absence of the SNP mutation in the nucleic acid sample can be detected. In the present embodiment, the probe which contains a complementary nucleotide to the SNP site may be a probe 45. In this case, the probe is designed such that it contains a complementary sequence to the one SNP nucleotide at its 3′-end.

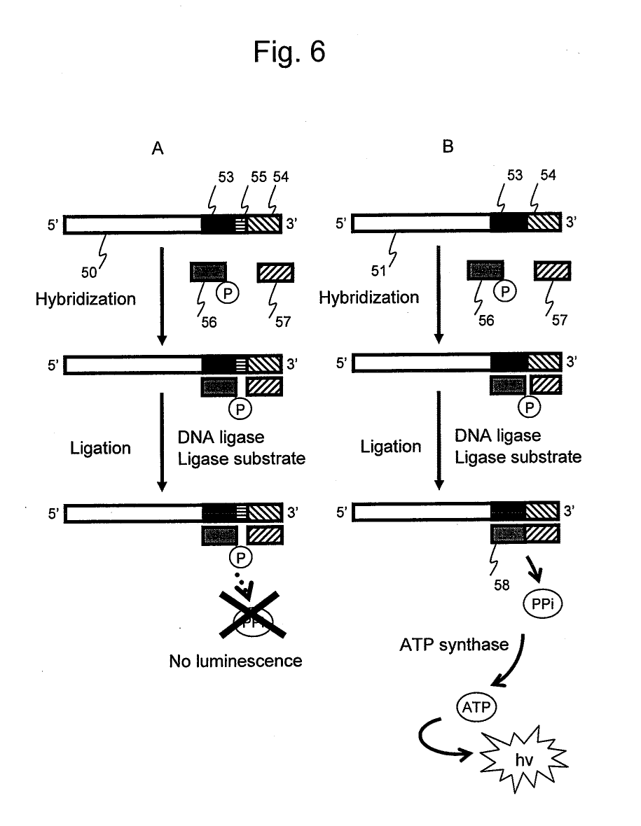

A fifth embodiment of the present invention is shown in FIG. 6. The present embodiment shows that at least two probes are hybridized to regions flanking upstream and downstream of a nucleotide sequence insertion site, respectively, in a nucleic-acid fragment. The present embodiment relates to a nucleotide analysis method comprising: a first step of hybridizing, to a single strand nucleic acid sample 50 containing a nucleic acid sequence 55 inserted between nucleic acid sequences 53 and 54 or a single strand nucleic acid sample 51 free from the nucleic acid sequence 55 inserted therebetween, a probe 56 which has a complementary sequence to the nucleic acid sequence 53 and has a 5′-end labeled with a phosphate group and a probe 57 which has a complementary sequence to the nucleic acid sequence 54; a second step of performing ligation reaction of the probes hybridized in the first step using ligase incorporating a ligase substrate; a third step of exchanging pyrophosphoric acid produced through the successful ligation reaction to ATP using ATP synthase; and a step of detecting chemiluminescence dependent on the obtained ATP. More specifically, the present embodiment relates to a method for detecting nucleotide sequence insertion (the presence or absence of a mutation in the nucleotide sequence insertion site) in the nucleic acid sample.

FIG. 6A shows the analysis of the single strand nucleic acid sample 50 containing nucleotide sequence insertion. FIG. 6B shows the analysis of the single strand nucleic acid sample 51 free from nucleotide sequence insertion. In FIG. 6A that shows nucleotide sequence insertion, a gap occurs between the probes hybridized in the first step. Therefore, a ligation product is not obtained in the second step. Accordingly, no pyrophosphoric acid is produced, and chemiluminescence is not detected. On the other hand, in FIG. 6B that is free from nucleotide sequence insertion, the probes are hybridized without a gap in the first step. Therefore, in the second step, ligation takes place using ligase to obtain a ligation product 58. Accordingly, pyrophosphoric acid is produced, and chemiluminescence is detected. Based on the presence or absence of this detectable chemiluminescence, the presence or absence of the nucleotide sequence insertion in the nucleic acid sample can be detected.

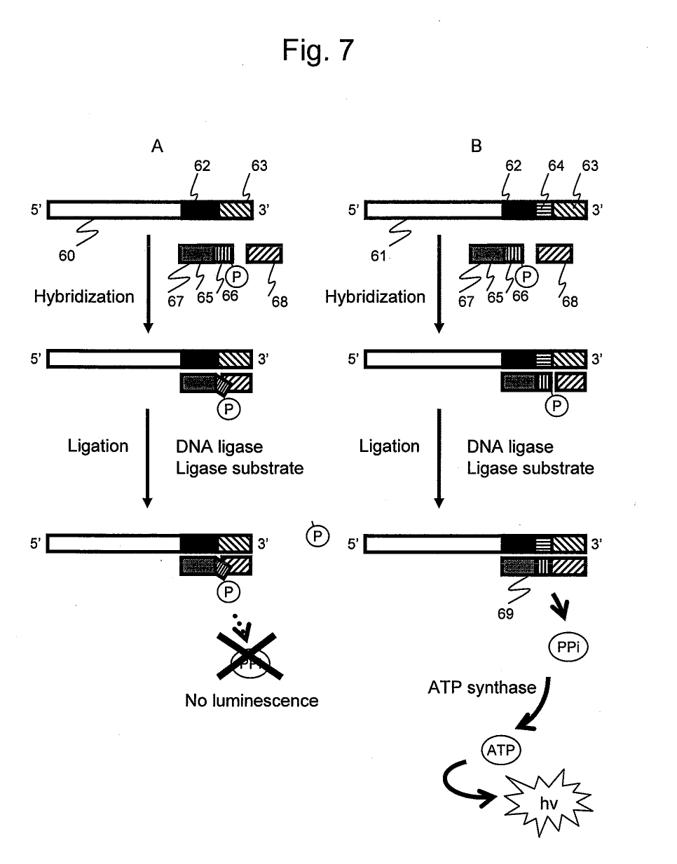

A sixth embodiment of the present invention is shown in FIG. 7. The present embodiment shows that at least one probe of at least two probes has an end (5′- or 3′-end sequence) corresponding to (complementary to) a nucleotide sequence deletion site in a nucleic-acid fragment (nucleic acid sample). The present embodiment relates to a nucleotide analysis method comprising: a first step of hybridizing, to a single strand nucleic acid sample 60 having deletion of a nucleic acid sequence 64 between nucleic acid sequences 62 and 63 or a single strand nucleic acid sample 61 free from deletion of the nucleic acid sequence 64 therebetween, a probe 67 which has nucleic acid sequences 65 and 66 complementary to the nucleic acid sequences 62 and 64 and has a 5′-end labeled with a phosphate group and a probe 68 which has a complementary sequence to the nucleic acid sequence 63; a second step of performing ligation reaction of the probes hybridized in the first step using ligase incorporating a ligase substrate; a third step of exchanging pyrophosphoric acid produced through the successful ligation reaction to ATP using ATP synthase; and a step of detecting chemiluminescence dependent on the obtained ATP. More specifically, the present embodiment relates to a method for detecting nucleotide sequence deletion (the presence or absence of a mutation in the nucleotide sequence deletion site).

FIG. 7A shows the analysis of the single strand nucleic acid sample 60 containing nucleotide sequence deletion. FIG. 7B shows the analysis of the single strand nucleic acid sample 61 free from nucleotide sequence deletion. In FIG. 7A that shows nucleotide sequence deletion, the nucleic acid sequence 66 contained at the 5′-end of the probe 67 hybridized in the first step takes a single strand form. Therefore, a ligation product is not obtained in the second step. Accordingly, no pyrophosphoric acid is produced, and chemiluminescence is not detected. On the other hand, in FIG. 7B that is free from nucleotide sequence deletion, the nucleic acid sequence 66 contained at the 5′-end of the probe 67 hybridized in the first step is hybridized to the nucleic acid sequence 64. Therefore, in the second step, ligation takes place using ligase to obtain a ligation product 69. Accordingly, pyrophosphoric acid is produced, and chemiluminescence is detected. Based on the presence or absence of this detectable chemiluminescence, the presence or absence of the nucleotide sequence deletion in the nucleic acid sample can be detected. In the present embodiment, the probe which contains a complementary sequence to the deletion sequence may be a probe 68. In this case, the probe is designed such that it contains a complementary sequence to the deletion sequence at its 3′-end.

### EXAMPLES

Hereinafter, the present invention will be described with reference to Examples. However, the present invention is not intended to be limited to these Examples.

### Example 1

The following synthetic oligo DNAs were used in Example 1:

Nucleic acid sample 1:

(SEQ ID NO: 1)

5′-CTCTCTCATCAGCGAACCACAACTCAAGACCTCGTTAAGGGAGCGGA
GCGGTAATGCTAGTTATTGTCCA-3′
Nucleic acid sample 2:

(SEQ ID NO: 2)

5′-CTCTCTCATCAGCGAACCACAACTCAAGACCTCGTTAAGGGAGCGGA
GCG-3′
Probe 1:

(SEQ ID NO: 3)

5′P-CGCTCCGCTCCCTTAACGAG-3′
Probe 2:

(SEQ ID NO: 4)

5′TET-TGGACAATAACTAGCATTAC-3′

In a first embodiment of the present invention, a reaction product was confirmed by electrophoresis to confirm whether the presence or absence of a nucleic acid sequence in a nucleic acid sample agrees with the presence or absence of a ligation product according to the present method.

The above-described synthetic oligo DNAs were used as nucleic acid samples and probes. The nucleic acid sample 1 has a 70-nt (nucleotide) sequence. The nucleic acid sample 2 has a 50-nt sequence obtained by deleting at 51 to 70 nucleotide positions from the 5′-end of the nucleic acid sample 1. The probe 1 is a probe of 20 nucleotides in base length which has a complementary sequence to the 31 to 50 nucleotide positions from the 5′-end of the nucleic acid sample 1 or 2 and has a 5′-end labeled with a phosphate group. The probe 2 is a probe of 20 nucleotides in base length which has a complementary sequence to the 51 to 70 nucleotide positions from the 5′-end of the nucleic acid sample 1 and has a 5′-end labeled with TET.

Ligase and a ligase substrate used were Pfu DNA ligase and dATPαS, respectively. The composition of reaction and luminescent reagents is shown in Tables 1 and 2.

TABLE 1
Composition of luminescent reagent

| Reagent | Final Concentration | |
|---|---|---|
| Tricine | 50.0 | mM |
| EDTA | 0.5 | mM |
| MgAc | 5.0 | mM |
| DTT | 0.5 | mM |
| PPDK | 33.8 | U/mL |
| Luciferase | 523.0 | GLU/mL |
| Apyrase | 0.9 × 10⁻³ | U/mL |
| Luciferin | 0.4 | mM |
| PEP/3Na | 0.8 × 10⁻¹ | U/mL |
| AMP | 0.4 | mM |

| | |
|---|---|
| BSA | 0.1 × 10⁻¹% |

TABLE 2
Composition of reaction reagent

| Reagent | Final Concentration |
|---|---|
| Tris-HCl (pH 7.5) | 20.0 mM |
| KCl | 20.0 mM |
| MgCl₂ | 10.0 mM |
| Isopal | 0.1% |
| DTT | 1 mM |

The luminescent reagent contains PPDK used in ATP production reaction and luciferase and luciferin used in luminescence reaction.

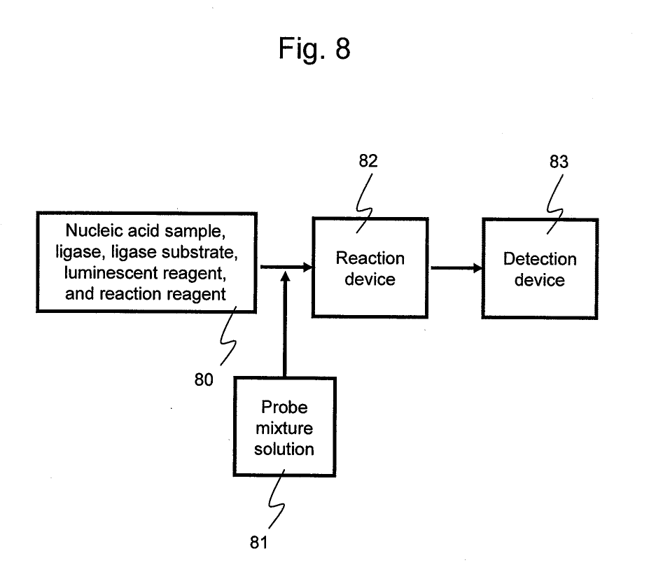

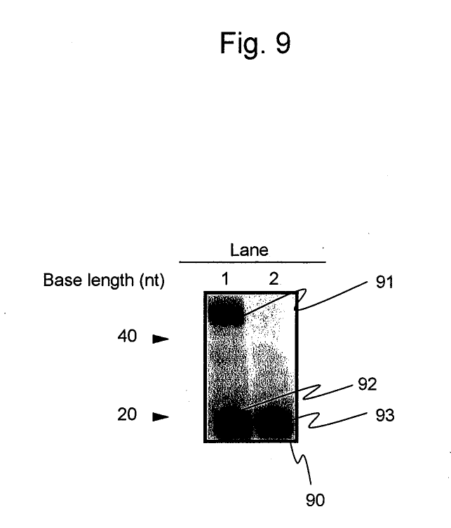

A reaction flow is shown in FIG. 8. 2 μL of a probe mixture solution 81 containing the probes 1 and 2 (100 μM each) was added to a reaction solution 80 containing the nucleic acid sample (final concentration: 0.25 the Pfu DNA ligase (final concentration: 0.05 U/μL), the dATPαS (final concentration: 0.65 mM), the luminescent reagent, and the reaction reagent. The mixture was reacted in a reaction device 82 at 40° C. for 1 hour. The reaction product was electrophoresed on an 8 M urea+15% acrylamide gel. Then, TET signals were detected from the acrylamide gel using a detection device 83. The reaction device 82 and the detection device 83 used were GeneAmp PCR System 9700 (Applied Biosystems) and FluorImager 595 (GE Healthcare), respectively. The electrophoresis results obtained by Example 1 are shown in an electrophoresis image 90 in FIG. 9. A lane 1 shows the reaction product derived from the nucleic acid sample 1. A lane 2 shows the reaction product derived from the nucleic acid sample 2. As a result, in the lane 1, a band 91 could be confirmed at a position of 40 nt corresponding to the base length of the ligation product. By contrast, in the lane 2, a band could not be confirmed at this position. Bands 92 and 93 at a 20-nt base length seen in both the lanes 1 and 2 are derived from the unligated probe 2 labeled with TET. This result can demonstrate that a ligation product is obtained using the nucleic acid sample 1 having two nucleic acid sequences, whereas no ligation product is obtained using the nucleic acid sample 2 having deletion of one of the nucleic acid sequences. Thus, it could be confirmed that in the first embodiment of the present invention, the presence or absence of a nucleic acid sequence in a nucleic acid sample agrees with the presence or absence of a ligation product.

### Example 2

The following synthetic oligo DNAs were used in Example 2:

Nucleic acid sample 1:

(SEQ ID NO: 1)

5′-CTCTCTCATCAGCGAACCACAACTCAAGACCTCGTTAAGGGAGCGGA
GCGGTAATGCTAGTTATTGTCCA-3′
Nucleic acid sample 2:

(SEQ ID NO: 2)

5′-CTCTCTCATCAGCGAACCACAACTCAAGACCTCGTTAAGGGAGCGGA
GCG-3′
Probe 1:

(SEQ ID NO: 3)

5′P-CGCTCCGCTCCCTTAACGAG-3′
Probe 2:

(SEQ ID NO: 4)

5′TET-TGGACAATAACTAGCATTAC-3′

In the first embodiment of the present invention, chemiluminescence attributed to a reaction product was detected to confirm whether the presence or absence of a nucleic acid sequence in a nucleic acid sample can be detected based on chemiluminescence.

The same nucleic acid samples, probes, and reaction composition as in Example 1 were used. To decrease a background in luminescence detection, a reaction solution was incubated at 40° C. for 1 hour to remove ligation reaction-underived ATP present in the reaction solution using apyrase. Then, the analysis was conducted.

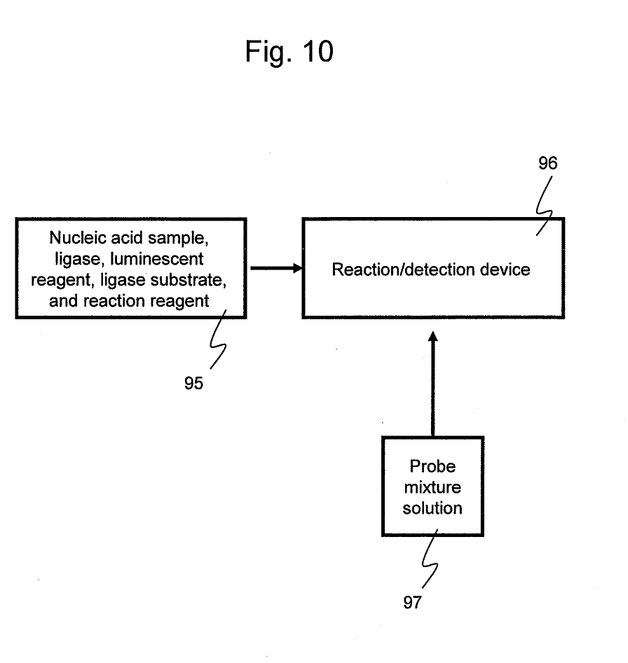

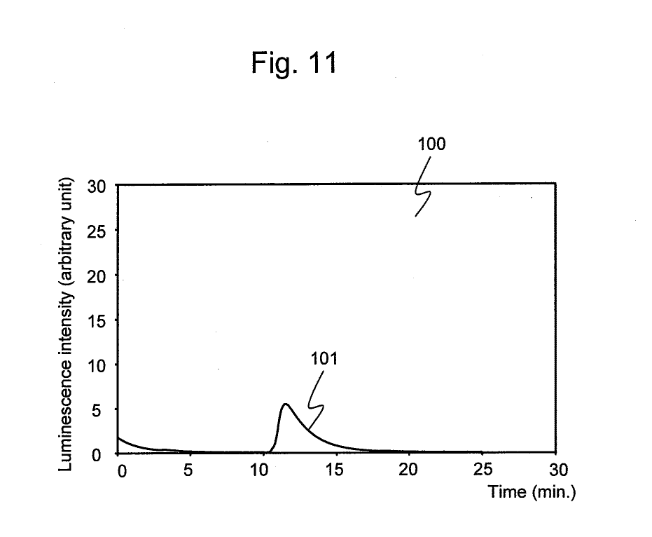

A reaction flow is shown in FIG. 10. A reaction solution 95 containing the nucleic acid sample (final concentration: 0.25 μM), the Pfu DNA ligase (final concentration: 0.05 U/μL), the dATPαS (final concentration: 0.65 mM), the luminescent reagent, and the reaction reagent was reacted in a reaction/detection device 96 at 40° C. for 1 hour. Then, luminescence intensity was detected for 10 minutes. Then, 2 μL of a probe mixture solution 97 containing the probes 1 and 2 (100 μM each) was added thereto, and luminescence intensity was detected for 20 minutes. The reaction/detection device 96 used was an automatic chemiluminometer. The luminescence intensity (signal intensity) observed by Example 2 is shown in a graph 100 in FIG. 11. A time-luminescence intensity curve (hereinafter, luminescence spectrum) 101 shows the results obtained from the reaction solution supplemented with the nucleic acid sample 1. A peak in the luminescence spectrum 101 was detected due to the probe addition 10 minutes after the start of luminescence spectrum detection. By contrast, a luminescence spectrum peak was not detected at the same time from the reaction solution supplemented with the nucleic acid sample 2. In consideration of the results of Example 1, this result demonstrates that in the first embodiment of the present invention, ligation reaction can be detected based on chemiluminescence. Thus, it could be confirmed that the presence or absence of a nucleic acid sequence can be detected based on chemiluminescence using the present invention.

### Example 3

The following synthetic oligo DNAs were used in Example 3:

| | |
|---|---|
| Primer 1: | |
| 5′-ATCCGGATATAGTTCCTCCTTTCAG-3′ | (SEQ ID NO: 5) |
| Primer 2: | |
| 5′-CCATCGCCGCTTCCACTTTTT-3′ | (SEQ ID NO: 6) |
| Probe 3: | |
| 5′P-CCAGTAGTAGGTTGAGGCCGTT-3′ | (SEQ ID NO: 7) |
| Probe 4: | |
| 5′-GACTCCTGCATTAGGAAGCAGC-3′ | (SEQ ID NO: 8) |

In a second embodiment of the present invention, chemiluminescence attributed to a reaction product was detected to confirm whether an amplified nucleic acid sample in analysis can be quantitatively detected based on chemiluminescence.

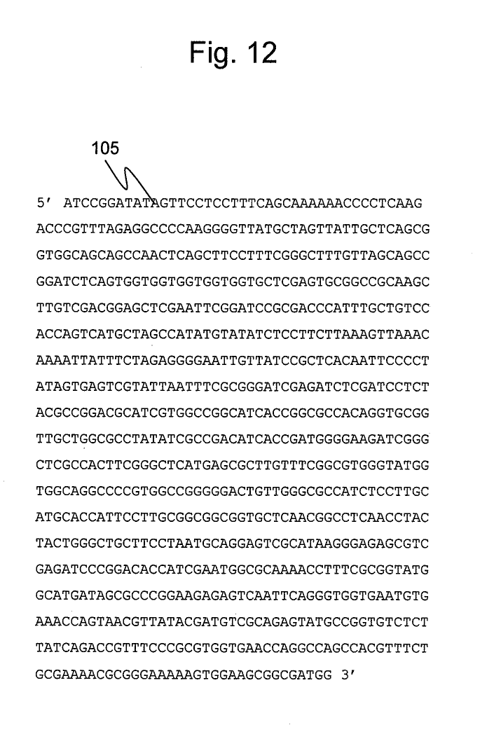

pET21a vector DNA (TAKARA BIO) prepared in 10³ copies and 10⁶ copies was used as nucleic acid samples. The above-described primers were used as oligonucleotide primers for amplification. A nucleotide sequence 105 (SEQ ID NO: 9) of an elongation product is shown in FIG. 12. The primer 1 is a forward primer having the same sequence as the 1st to 25 nucleotide positions from the 5′-end of the nucleotide sequence 105 described in FIG. 12. The primer 2 is a reverse primer having a complementary sequence to the 820 to 840 nucleotide positions. Polymerase and a PCR reaction reagent used were Pfu DNA polymerase (STRATAGENE) and a buffer included with the polymerase, respectively. The amount of the enzyme used and the amounts of dNTP and the primers followed a manual included with the enzyme. A PCR product obtained as an elongation product was purified, for use, by gel filtration using Sephadex G100 to remove the primers and the dNTPs.

Next, the probes 3 and 4 were used as oligonucleotide probes hybridized to the elongation product. The probe 3 has a complementary sequence to the 566 to 587 nucleotide positions from the 5′-end of the nucleotide sequence 105 described in FIG. 12 and has a 5′-end labeled with a phosphate group. The probe 4 has a complementary sequence to the 588 to 609 nucleotide positions. Ligase and a ligase substrate used were Pfu DNA ligase and dATPαS, respectively. The same composition of luminescent and reaction reagents as in Tables 1 and 2 was used. The luminescent reagent contains PPDK used in ATP production reaction and luciferase and luciferin used in luminescence reaction. As in Example 2, to necessarily degrade, in advance, ligation reaction-underived ATP present in the reaction solution, a reaction solution was incubated at 40° C. for 1 hour to remove the ATP in the reaction solution using apyrase. Then, the analysis was conducted.

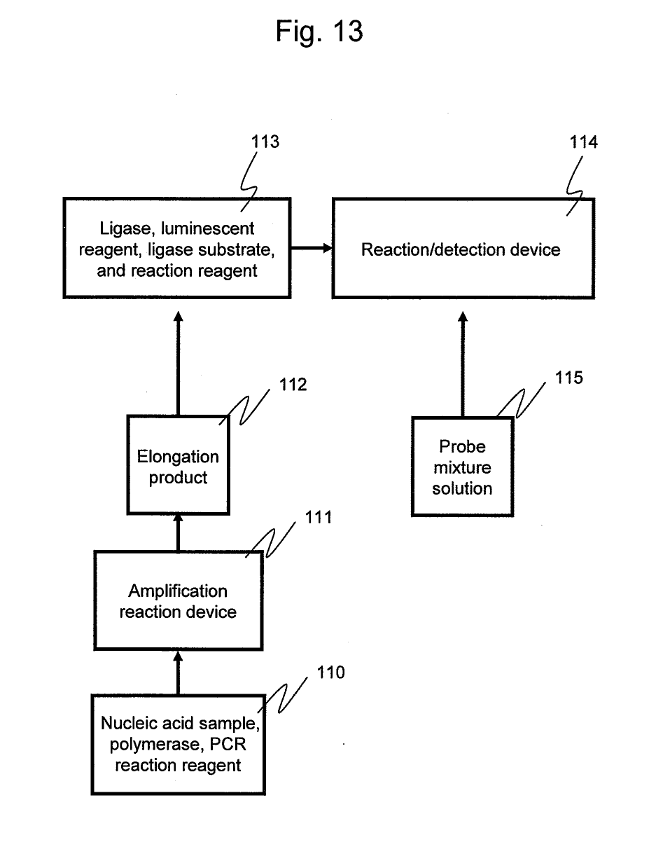

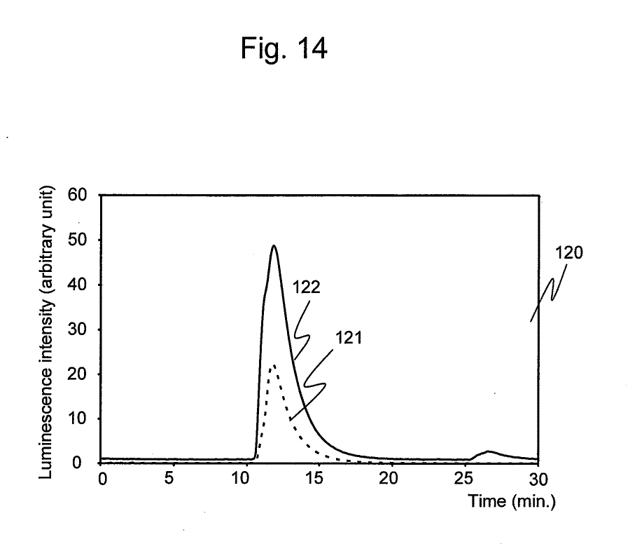

A reaction flow is shown in FIG. 13. A reaction solution 110 containing the nucleic acid sample, the Pfu DNA polymerase (final concentration: 0.05 U/μL), and the PCR reaction reagent was added to an amplification reaction device 111 to thereby perform PCR reaction. An elongation product 112 (amplified nucleic-acid fragment) obtained through the PCR reaction was added to a reaction solution 113 containing the Pfu DNA ligase (final concentration: 0.05 U/μL), the dATPαS (final concentration: 0.65 mM), the luminescent reagent, and the reaction reagent. The mixture was reacted in a reaction/detection device 114 at 40° C. for 1 hour. Then, luminescence intensity was detected for 10 minutes. Then, 2 of a probe mixture solution 115 containing the probes 3 and 4 (100 μM each) was added thereto, and luminescence intensity was detected for 20 minutes. The amplification reaction device 111 and the reaction/detection device 114 used were GeneAmp PCR System 9700 (Applied Biosystems) and an automatic chemiluminometer, respectively. The luminescence intensity (signal intensity) observed by Example 3 is shown in a graph 120 in FIG. 14. A luminescence spectrum 121 shows the results obtained from 10³ copies of the nucleic acid sample. A luminescence spectrum 122 shows the results obtained from 10⁶ copies of the nucleic acid sample. As a result, a peak was detected in both the luminescence spectra 121 and 122 due to the probe addition 10 minutes after the start of luminescence spectrum detection. The quantitative ratio of luminescence intensity between the peaks of the luminescence spectra 121 and 122 is approximately 1:2. This value almost agrees with the ratio between the copy numbers of the nucleic acid sample. These results demonstrate that in the second embodiment of the present invention, the amount of luminescence detected is increased depending on the copy number of a nucleic acid sample used in the form of an elongation product as the nucleic acid sample in the detection. Thus, it was confirmed that a nucleic acid sample can be quantitatively detected using the present invention.

### Example 4

The following synthetic oligo DNAs were used in Example 4:

Nucleic acid sample 3:

(SEQ ID NO: 10)

5′-AAAAAAAAAAAAAAAAAAAAAAAAAAAAAAAAAAAAAAAAAAAAAAA
AAAAAAAAAAAAA-3′
Probe 5:

(SEQ ID NO: 11)

5′P-TTTTTTTTTTTTTTTTTTTT-3′

In a third embodiment of the present invention, the amount of chemiluminescence attributed to a reaction product was detected to confirm whether the polyA length of mRNA used as a nucleic acid sample can be detected based on chemiluminescence.

The nucleic acid sample used was obtained by: transcribing, using T7 RNA polymerase (Invitrogen), RNA from a construct having the core region of hepatitis C virus (HCV) type 1a; reacting the RNA with Poly(A) Polymerase (TAKARA) for 30 or 70 minutes to add polyA thereto; confirming the polyA length of 40 or 80 nt by electrophoresis; and purifying the reaction product. The reaction composition of the RNA transcription and the polyA addition followed protocols included with the enzymes.

The nucleic acid sample 3 having a 60-nt polyA sequence was used as a control for the polyA length. Moreover, the probe 5 was used as a probe hybridized to the nucleic acid sample. The probe 5 has a 20-nt dT sequence and has a 5′-end labeled with a phosphate group.

Ligase and a ligase substrate used were Pfu DNA ligase and dATPαS, respectively. The composition of luminescent and reaction reagents is shown in Tables 3 and 4.

TABLE 3
Composition of luminescent reagent

| Reagent | Final Concentration | |
|---|---|---|
| Tricine | 60.0 | mM |
| EDTA | 2.0 | mM |
| MgAc | 20.0 | mM |
| DTT | 0.2 | mM |
| ATP sulfurylase | 0.2 | U/mL |
| Luciferase | 50.0 | GLU/mL |
| Apyrase | 0.5 × 10⁻³ | U/mL |
| Luciferin | 0.4 | mM |
| APS | 5 | uM |

| | |
|---|---|
| BSA | 0.1% |

TABLE 4
Composition of reaction reagent

| Reagent | Final Concentration | |
|---|---|---|
| Tris-HCl (pH 7.5) | 20.0 | mM |
| MgCl₂ | 10.0 | mM |
| Tween20 | 0.1% | |
| BSA | 0.1 | mg/ml |

The luminescent reagent contains ATP sulfurylase used in ATP production reaction and luciferase and luciferin used in luminescence reaction. As in Examples 2 and 3, a reaction solution was incubated in advance at 40° C. for 1 hour to remove ATP in the reaction solution. Then, the analysis was conducted.

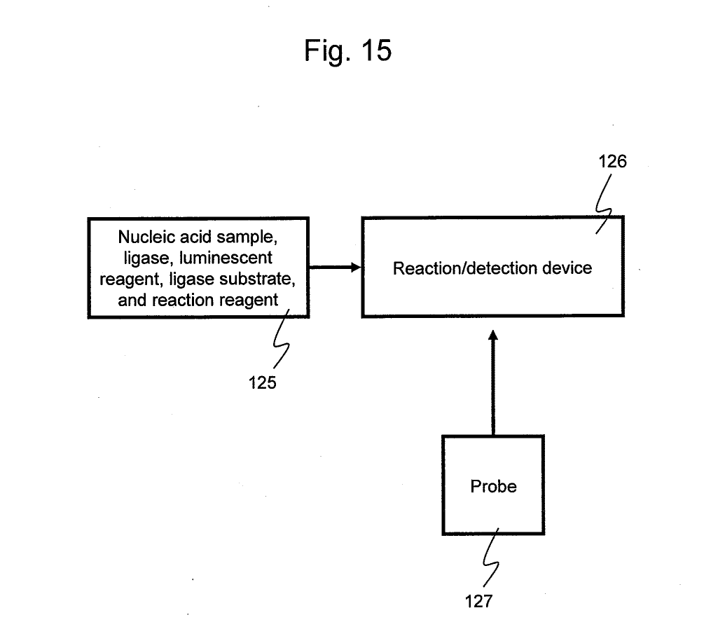

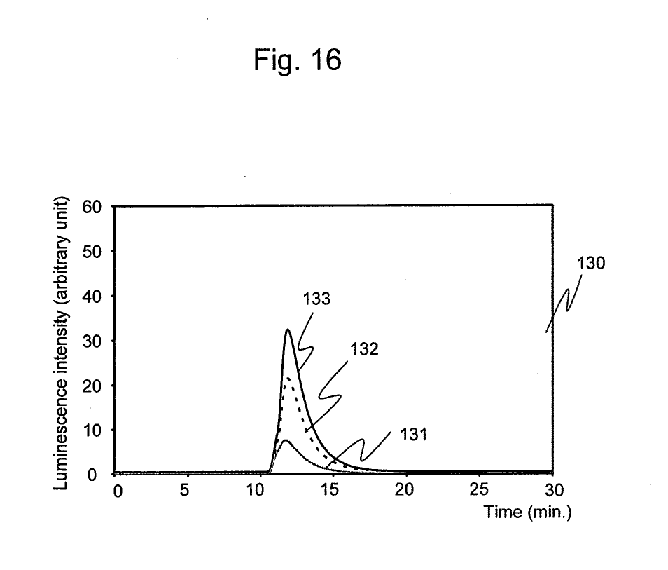

A reaction flow is shown in FIG. 15. A reaction solution 125 containing the nucleic acid sample (final concentration: 0.5 μM), the Pfu DNA ligase (final concentration: 0.1 U/μL), the dATPαS (final concentration: 1.0 mM), the luminescent reagent, and the reaction reagent was reacted in a reaction/detection device 126 at 40° C. for 1 hour. Then, luminescence intensity was detected for 10 minutes. Then, 1 μL of a probe 127 (100 μM) was added thereto, and luminescence intensity was detected for 20 minutes. The reaction/detection device 126 used was an automatic chemiluminometer. The luminescence intensity (signal intensity) observed in the present Example is shown in a graph 130 in FIG. 16. A luminescence spectrum 131 shows the analysis results obtained using the nucleic acid sample containing the 40-nt polyA added. A luminescence spectrum 132 shows the analysis results obtained using the nucleic acid sample 3 having the 60-nt polyA sequence. A luminescence spectrum 133 shows the analysis results obtained using the nucleic acid sample containing the 80-nt polyA added. A peak was detected in all the spectra due to the probe addition 10 minutes after the start of luminescence detection. The quantitative ratio of luminescence intensity among the peaks of the luminescence spectra 131, 132, and 133 is approximately 1:2:3. This value agrees with the ratio among the analyzed polyA lengths. These results demonstrate that in the third embodiment of the present invention, the amount of luminescence detected is increased in proportion to the number of ligated sites in polyA length detection. Thus, it was confirmed that a polyA length can be detected using the present invention.

### Example 5

The following synthetic oligo DNAs were used in Example 5:

| | |
|---|---|
| Primer 3: | |
| 5′-GACCAGTAAGTTCAGAGATGCAGA-3′ | (SEQ ID NO: 12) |
| Primer 4: | |
| 5′-CAACCAGACCAGGTAGACAGAG-3′ | (SEQ ID NO: 13) |
| Probe 6: | |
| 5′P-TGTTCTCACCGATACACTTC-3′ | (SEQ ID NO: 14) |
| Probe 7: | |
| 5′-AAAGACCTCCCAGCGGCCAA-3′ | (SEQ ID NO: 15) |

In a fourth embodiment of the present invention, chemiluminescence attributed to a reaction product was detected to confirm whether the presence or absence of a mutation in a nucleic acid sample containing SNP can be detected by the method of the present invention.

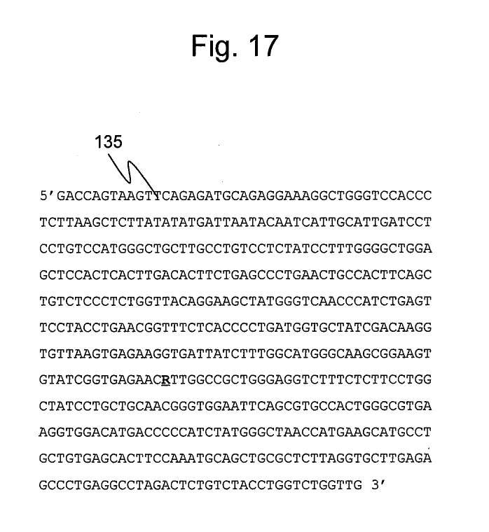

The nucleic acid sample used was a CYP1A1 gene region (Accession No. X02612) amplified by PCR from the genome purified from blood provided by a volunteer. The genome purification procedure followed Molecular Cloning, Second edition (Cold Spring Harbor Laboratory Press, 1989), unless otherwise specified. An amplified product used as the nucleic acid sample was purified by gel filtration using Sephadex G100 to remove the primers and the dNTPs. A nucleotide sequence 135 (SEQ ID NO: 16) of the region amplified by PCR is shown in FIG. 17. A 328th nucleotide R from the 5′-end of the nucleotide sequence of the amplified product used as the nucleic acid sample has SNP and is substituted by A or G. The nucleic acid sample used in the analysis was sequenced in advance for SNP identification to thereby confirm the nucleotide substitutions of these two kinds. The above-described synthetic oligo DNAs were used as PCR primers and probes. The primer 3 is a forward primer having the same sequence as the 1st to 24 nucleotide positions from the 5′-end of the nucleotide sequence 135 described in FIG. 17. The primer 4 is a reverse primer having a complementary sequence to the 509 to 530 nucleotide positions. The probe 6 has a complementary sequence to the 309 to 328 nucleotide positions and has a 5′-end labeled with a phosphate group. The probe 6 recognizes the SNP sequence A. The probe 7 has a complementary sequence to the 329 to 348 nucleotide positions.

Ligase and a ligase substrate used were Pfu DNA ligase and dATPαS, respectively. The same composition of luminescent and reaction reagents as in Tables 3 and 4 was used. The luminescent reagent contains ATP sulfurylase used in ATP production reaction and luciferase and luciferin used in luminescence reaction. As in Examples 2, 3, and 4, a reaction solution was incubated in advance at 40° C. for 1 hour to remove ATP in the reaction solution. Then, the analysis was conducted.

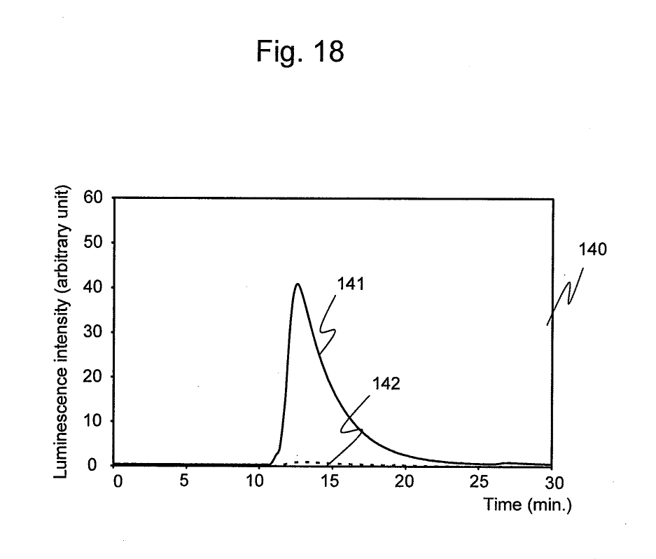

A reaction flow is shown in FIG. 10. A reaction solution 95 containing the nucleic acid sample (final concentration: 0.5 μM), the Pfu DNA ligase (final concentration: 0.1 U/μL), the dATPαS (final concentration: 1.0 mM), the luminescent reagent, and the reaction reagent was reacted in a reaction/detection device 96 at 40° C. for 1 hour. Then, luminescence intensity was detected for 10 minutes. Then, 2 μL of a probe mixture solution 97 containing the probes 6 and 7 (100 μM each) was added thereto, and luminescence intensity was detected for 20 minutes. The reaction/detection device 96 used was an automatic chemiluminometer. The luminescence intensity (signal intensity) observed in the present Example is shown in a graph 140 in FIG. 18. A luminescence spectrum 141 shows the results obtained using the nucleic acid sample having the SNP site A. A peak was detected in the luminescence spectrum 141 due to the probe addition 10 minutes after the start of luminescence detection. A luminescence spectrum 142 shows the results obtained using the nucleic acid sample having the SNP site G. A peak was not detected in the luminescence spectrum 142. From these results, it was confirmed that the presence or absence of a SNP mutation in a nucleic acid sample can be detected based on luminescence using the present invention.

### Example 6

The following synthetic oligo DNAs were used in Example 6:

Primer 5:

(SEQ ID NO: 17)

5′-TGTGTGACCTAACTGTGTAA-3′
Primer 6:

(SEQ ID NO: 18)

5′-ACCTTCCCACTAGAGCTTGG-3′
Probe 8:

(SEQ ID NO: 19)

5′P-TAATCTATTACACTTTATATTACCCATTAT-3′
Probe 9:

(SEQ ID NO: 20)

5′-GGTTTCTTTTCTCTCTCCCACCCACAACTA-3′

In a fifth embodiment of the present invention, chemiluminescence attributed to a reaction product was detected to confirm whether the presence or absence of a mutation (nucleotide sequence insertion) in a nucleic acid sample can be detected by the method of the present invention.

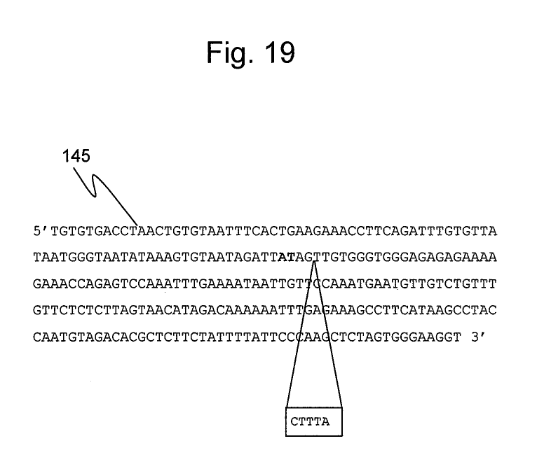

The nucleic acid sample used was a 3′ non-translated region (Accession No. U59263) of a leptin receptor gene amplified by PCR from the genome purified from blood provided by a volunteer. The genome purification procedure followed Molecular Cloning, Second edition (Cold Spring Harbor Laboratory Press, 1989), unless otherwise specified. An amplified product used as the nucleic acid sample was purified by gel filtration using Sephadex G100 to remove the primers and the dNTPs. A nucleotide sequence 145 (SEQ ID NO: 21) of the region amplified by PCR is shown in FIG. 19. It is known that the nucleotide sequence insertion of CTTTA between the 80th adenine and the 81st thymine from the 5′-end of the nucleotide sequence 145 occurs depending on susceptibility to a disease associated with a low HDL (high-density lipoprotein) cholesterol concentration. The nucleic acid sample used in the analysis was sequenced in advance to thereby confirm the presence or absence of the nucleotide sequence insertion. The above-described synthetic oligo DNAs were used as PCR primers and probes. The primer 5 is a forward primer having the same sequence as the 1st to 20 nucleotide positions from the 5′-end of the nucleotide sequence 145 described in FIG. 19. The primer 6 is a reverse primer having a complementary sequence to the 240 to 259 nucleotide positions. The probe 8 has a complementary sequence to the 51 to 80 nucleotide positions and has a 5′-end labeled with a phosphate group. The probe 9 has a complementary sequence to the 81 to 110 nucleotide positions.

Ligase and a ligase substrate used were Pfu DNA ligase and dATPαS, respectively. The same composition of luminescent and reaction reagents as in Tables 1 and 2 was used. The luminescent reagent contains PPDK used in ATP production reaction and luciferase and luciferin used in luminescence reaction. As in Examples 2, 3, 4, and 5, a reaction solution was incubated in advance at 40° C. for 1 hour to remove ATP in the reaction solution. Then, the analysis was conducted.

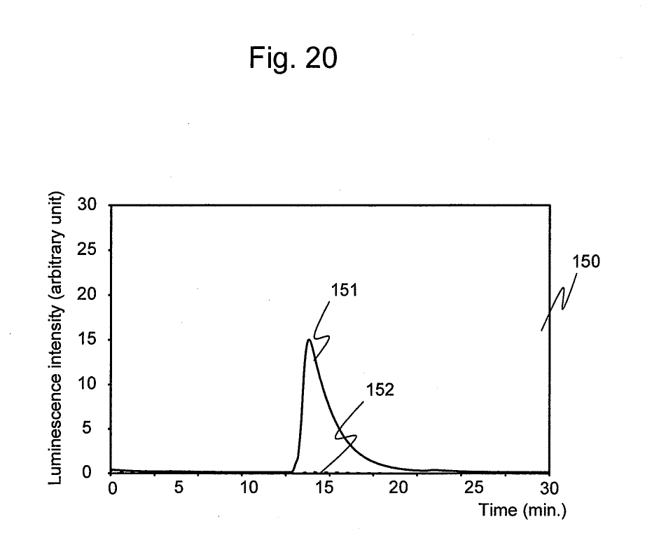

A reaction flow is shown in FIG. 10. A reaction solution 95 containing the nucleic acid sample (final concentration: 0.3 μM), the Pfu DNA ligase (final concentration: 0.05 U/μL), the dATPαS (final concentration: 0.7 mM), the luminescent reagent, and the reaction reagent was reacted in a reaction/detection device 96 at 40° C. for 1 hour. Then, luminescence intensity was detected for 10 minutes. Then, 2 μL of a probe mixture solution 97 containing the probes 8 and 9 (100 μM each) was added thereto, and luminescence intensity was detected for 20 minutes. The reaction/detection device 96 used was an automatic chemiluminometer. The luminescence intensity (signal intensity) observed in the present Example is shown in a graph 150 in FIG. 20. A luminescence spectrum 151 shows the results obtained using the nucleic acid sample free from the nucleotide sequence insertion. A peak was detected in the luminescence spectrum 151 due to the probe addition 10 minutes after the start of luminescence detection. A luminescence spectrum 152 shows the results obtained using the nucleic acid sample having the nucleotide sequence insertion. A peak was not detected in the luminescence spectrum 152.

From these results, it was confirmed that the presence or absence of nucleotide sequence insertion in a nucleic acid sample can be analyzed by luminescence detection using the present invention.

### Example 7

The following synthetic oligo DNAs were used in Example 7:

Primer 7:

(SEQ ID NO: 22)

5′-AAGCGCACGCTGCGGAGGCTGCTG-3′
Primer 8:

(SEQ ID NO: 23)

5′-GGCTGCCAGGTCGCGGTGCA-3′
Probe 10:

(SEQ ID NO: 24)

5′P-GCTTCTCTTAATTCCTTGATAGCGACGGGA-3′
Probe 11:

(SEQ ID NO: 25)

5′-GAGGATTTCCTTGTTGGCTTTCGGAGATGTT-3′

In a sixth embodiment of the present invention, chemiluminescence attributed to a reaction product was detected to confirm whether the presence or absence of a mutation (nucleotide sequence deletion) in a nucleic acid sample can be detected by the method of the present invention.

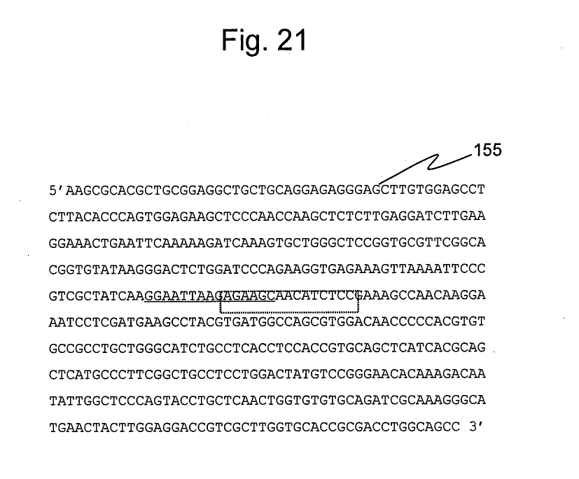

The nucleic acid sample used was an epidermal growth factor receptor (EGFR)-encoding gene region (Accession No. NM_{—}005228.3) amplified by PCR from the genome purified from blood provided by a volunteer. The genome purification procedure followed Molecular Cloning, Second edition (Cold Spring Harbor Laboratory Press, 1989), unless otherwise specified. An amplified product used as the nucleic acid sample was purified by gel filtration using Sephadex G100 to remove the primers and the dNTPs. A nucleotide sequence 155 (SEQ ID NO: 26) of the region amplified by PCR is shown in FIG. 21. It is known that the presence or absence of 15-nt deletion at the 210 to 224 nucleotide positions from the 5′-end of the nucleotide sequence 155 is effective for assessment on efficacy of gefitinib serving as an anticancer agent. The nucleic acid sample used in the analysis was sequenced in advance to thereby confirm the presence or absence of the nucleotide sequence deletion. The above-described synthetic oligo DNAs were used as PCR primers and probes. The primer 7 is a forward primer having the same sequence as the 1st to 24 nucleotide positions from the 5′-end of the nucleotide sequence 155. The primer 8 is a reverse primer having a complementary sequence to the 476 to 495 nucleotide positions. The probe 10 has a complementary sequence to the 195 to 224 nucleotide positions and has a 5′-end labeled with a phosphate group. The probe 11 has a complementary sequence to the 225 to 254 nucleotide positions.

Ligase and a ligase substrate used were Pfu DNA ligase and dATPαS, respectively. The same composition of luminescent and reaction reagents as in Tables 3 and 4 was used. The luminescent reagent contains ATP sulfurylase used in ATP production reaction and luciferase and luciferin used in luminescence reaction. As in Examples 2, 3, 4, 5, and 6, a reaction solution was incubated in advance at 40° C. for 1 hour to remove ATP in the reaction solution. Then, the analysis was conducted.

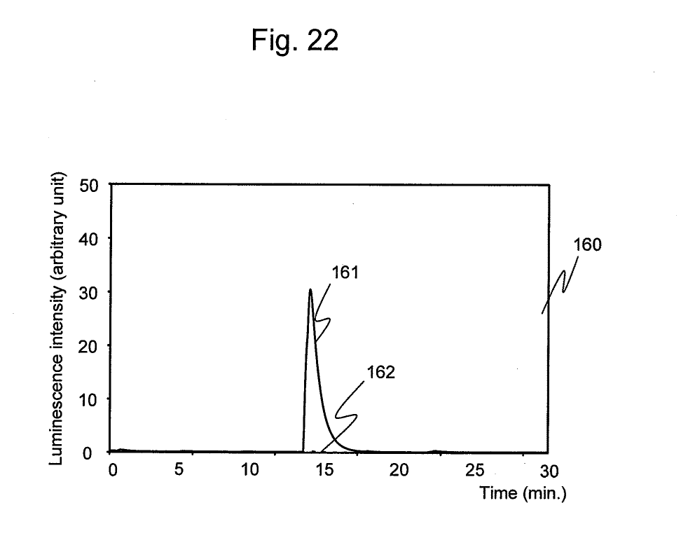

A reaction flow is shown in FIG. 10. A reaction solution 95 containing the nucleic acid sample (final concentration: 0.5 μM), the Pfu DNA ligase (final concentration: 0.1 U/μL), the dATPαS (final concentration: 1.0 mM), the luminescent reagent, and the reaction reagent was reacted in a reaction/detection device 96 at 40° C. for 1 hour. Then, luminescence intensity was detected for 10 minutes. Then, 2 μL of a probe mixture solution 97 containing the probes 10 and 11 (100 μM each) was added thereto, and luminescence intensity was detected for 20 minutes. The reaction/detection device 96 used was an automatic chemiluminometer. The luminescence intensity (signal intensity) observed in the present Example is shown in a graph 160 in FIG. 22. A luminescence spectrum 161 shows the results obtained using an amplified product free from the nucleotide sequence deletion as the nucleic acid sample. A peak was detected in the luminescence spectrum 161 due to the probe addition 10 minutes after the start of luminescence detection. A luminescence spectrum 162 shows the results obtained using an amplified product having the nucleotide sequence deletion as the nucleic acid sample. A peak was not detected in the luminescence spectrum 162. From these results, it could be confirmed that the presence or absence of nucleotide sequence deletion can be analyzed by luminescence detection using the present invention.

### FREE TEXT OF SEQUENCE LISTING

SEQ ID NO: 1—Description of artificial sequence: nucleic acid sample which is used in Example 1 of the present invention

SEQ ID NO: 2—Description of artificial sequence: nucleic acid sample which is used in Example 1 of the present invention

SEQ ID NO: 3—Description of artificial sequence: probe hybridized to nucleic acid sample, which is used in Example 1 of the present invention

SEQ ID NO: 4—Description of artificial sequence: probe hybridized to nucleic acid sample, which is used in Example 1 of the present invention

SEQ ID NO: 5—Description of artificial sequence: forward primer for amplifying nucleic acid sample, which is used in Example 3 of the present invention

SEQ ID NO: 6—Description of artificial sequence: reverse primer for amplifying nucleic acid sample, which is used in Example 3 of the present invention

SEQ ID NO: 7—Description of artificial sequence: probe hybridized to nucleic acid sample, which is used in Example 3 of the present invention

SEQ ID NO: 8—Description of artificial sequence: probe hybridized to nucleic acid sample, which is used in Example 3 of the present invention

SEQ ID NO: 9—Description of artificial sequence: nucleic acid sample sequence which is used in Example 3 of the present invention

SEQ ID NO: 10—Description of artificial sequence: nucleic acid sample having polyA sequence, which is used in Example 4 of the present invention

SEQ ID NO: 11—Description of artificial sequence: probe hybridized to polyA sequence, which is used in Example 4 of the present invention

SEQ ID NO: 12—Description of artificial sequence: forward primer for amplifying CYP1A1 gene region, which is used in Example 5 of the present invention

SEQ ID NO: 13—Description of artificial sequence: reverse primer for amplifying CYP1A1 gene region, which is used in Example 5 of the present invention

SEQ ID NO: 14—Description of artificial sequence: probe hybridized to CYP1A1 gene region, which is used in Example 5 of the present invention

SEQ ID NO: 15—Description of artificial sequence: probe hybridized to CYP1A1 gene region, which is used in Example 5 of the present invention

SEQ ID NO: 16—Description of artificial sequence: CYP1A1 gene sequence, which is used in Example 5 of the present invention

SEQ ID NO: 17—Description of artificial sequence: forward primer for amplifying leptin receptor gene region, which is used in Example 6 of the present invention

SEQ ID NO: 18—Description of artificial sequence: reverse primer for amplifying leptin receptor gene region, which is used in Example 6 of the present invention

SEQ ID NO: 19—Description of artificial sequence: probe hybridized to leptin receptor gene region, which is used in Example 6 of the present invention

SEQ ID NO: 20—Description of artificial sequence: probe hybridized to leptin receptor gene region, which is used in Example 6 of the present invention

SEQ ID NO: 21—Description of artificial sequence: leptin receptor gene sequence, which is used in Example 6 of the present invention

SEQ ID NO: 22—Description of artificial sequence: forward primer for amplifying EGFR gene region, which is used in Example 7 of the present invention

SEQ ID NO: 23—Description of artificial sequence: reverse primer for amplifying EGFR gene region, which is used in Example 7 of the present invention

SEQ ID NO: 24—Description of artificial sequence: probe hybridized to EGFR gene region, which is used in Example 7 of the present invention

SEQ ID NO: 25—Description of artificial sequence: probe hybridized to EGFR gene region, which is used in Example 7 of the present invention

SEQ ID NO: 26—Description of artificial sequence: EGFR gene sequence, which is used in Example 7 of the present invention

What is claimed is:

1. A nucleotide analysis method, comprising: hybridizing at least two probes to a nucleic-acid fragment; ligating the at least two probes using ligase; exchanging, to ATP, pyrophosphoric acid produced through the ligation reaction; and detecting chemiluminescence reaction dependent on the ATP.

2. The nucleotide analysis method according to claim 1, wherein the at least two probes are hybridized to adjacent regions, respectively, in the nucleic-acid fragment.

3. The nucleotide analysis method according to claim 1, wherein at least one probe of the at least two probes has a 5′-end labeled with a phosphate group.

4. The nucleotide analysis method according to claim 1, wherein the ligase catalyzes the ligation reaction using a substrate, and the chemiluminescence reaction is catalyzed by luciferase, wherein the substrate is substantially unreactive with the luciferase.

5. The nucleotide analysis method according to claim 1, wherein the ligase is capable of catalyzing the ligation reaction using the substrate which is substantially unreactive with the luciferase.

6. The nucleotide analysis method according to claim 1, wherein the chemiluminescence reaction is detected to thereby detect the presence, absence and/or amount of the sequence of interest in the nucleic-acid fragment.

7. The nucleotide analysis method according to claim 1, wherein the at least two probes are hybridized to RNA or DNA sequence regions, respectively, in the nucleic-acid fragment.

8. The nucleotide analysis method according to claim 1, wherein the at least two probes are hybridized to an amplified nucleic-acid fragment as the nucleic-acid fragment.

9. The nucleotide analysis method according to claim 1, wherein the at least two probes each comprise an oligo dT nucleotide.

10. The nucleotide analysis method according to claim 9, wherein the chemiluminescence reaction is detected to thereby measure the length of the nucleic-acid fragment.

11. The nucleotide analysis method according to claim 1, wherein the at least two probes are hybridized to direct repeat sequence regions, respectively, in the nucleic-acid fragment.

12. The nucleotide analysis method according to claim 11, wherein the direct repeat sequence in the nucleic-acid fragment is a particular nucleotide sequence occurring repetitively.

13. The nucleotide analysis method according to claim 11, wherein the at least two probes each comprise a complementary sequence to the direct repeat sequence.

14. The nucleotide analysis method according to claim 11, wherein the chemiluminescence reaction is detected to thereby measure the number of repetition of the direct repeat sequence.

15. The nucleotide analysis method according to claim 1, wherein at least one probe of the at least two probes has an end corresponding to an SNP site in the nucleic-acid fragment.

16. The nucleotide analysis method according to claim 15, wherein the chemiluminescence reaction is detected to thereby determine the presence or absence of the ligation reaction, based on which the presence or absence of a mutation in the SNP site is determined.

17. The nucleotide analysis method according to claim 1, wherein the at least two probes are hybridized to regions flanking upstream and downstream of a nucleotide sequence insertion site, respectively, in the nucleic-acid fragment.

18. The nucleotide analysis method according to claim 17, wherein the chemiluminescence reaction is detected to thereby determine the presence or absence of the ligation reaction, based on which the presence or absence of a mutation in the nucleotide sequence insertion site is determined.

19. The nucleotide analysis method according to claim 1, wherein at least one probe of the at least two probes has an end corresponding to a nucleotide sequence deletion site in the nucleic-acid fragment.

20. The nucleotide analysis method according to claim 19, wherein the chemiluminescence reaction is detected to thereby determine the presence or absence of the ligation reaction, based on which the presence or absence of a mutation in the nucleotide sequence deletion site is determined.
